(19) United States
(12) Patent Application Publication
(10) Pub. No.: US 2007/0250015 A1
(43) Pub. Date: Oct. 25, 2007

(54) Safety syringe with a retractable needle hub

(76) Inventors: Chien-Yuan Lee, Tamsui Town (TW); Chih-Cheng Kao, Taipei (TW)

Correspondence Address: ANDREWS KURTH LLP, 1350 I STREET, N.W., SUITE 1100, WASHINGTON, DC, 20005, US

(21) Appl. No.: 11/409,075

(22) Filed: Apr. 24, 2006

(51) Int. Cl. A61M 5/32

(52) U.S. Cl. 604/195

(57) ABSTRACT

A safety syringe with a retractable needle has a syringe body, a needle hub, a bolt and a plunger. The syringe body has a through hole. The needle hub is mounted in the syringe body and has a base. The base has a rear end, a front recess and a first connector. The first connector is formed in the rear end. The bolt with a cutout is mounted in the through hole and the front recess. The plunger is movably mounted in the syringe body and has a front plate and a base. The base is formed on the front plate and has a second connector. After the second connector is engaged with the first connector, the needle hub can be pushed forward to break the cutout. Then the plunger is pulled to allow the needle hub to be retracted into the syringe body.

### BACKGROUND OF THE INVENTION

1. Field of the Invention

The present invention relates to a syringe, and especially to a safety syringe for protecting a user from being pricked with a used needle.

2. Description of the Related Art

With modern developments in medical technology, how to protect a used needle from pricking a person is an important task.

The government states that used needles must be discarded carefully. However, a used needle easily pricks people when a needle cover is placed over the used needle.

### SUMMARY OF THE INVENTION

The objective of the present invention is to provide a safety syringe with a retractable needle hub that can keep a used needle from pricking a person when a protective cap is placed over the used needle.

To achieve the foregoing objective, a safety syringe with a retractable needle has a syringe body, a needle hub, a bolt and a plunger. The syringe body has a through hole, an inner surface and a limit protruding on the inner surface. The needle hub is mounted in the syringe body and has a base. The base has a rear end, a front recess and a first connector. The first connector is formed in the rear end. The bolt with a cutout is mounted in the through hole and the front recess. The plunger is movably mounted in the syringe body and has a front plate and a base. The base is formed on the front plate and has a second connector formed on the base. After the second connector is engaged with the first connector, the needle hub can be pushed forward to break the cutout. Then the plunger is pulled to allow the needle hub with the needle to be retracted into the syringe body.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a safety syringe with a retractable needle hub in accordance with the present invention;

FIG. 2 is an exploded perspective view of the first embodiment of the safety syringe with the retractable needle hub in FIG. 1;

FIG. 3 is an exploded perspective view of the first embodiment of the safety syringe with the retractable needle hub in FIG. 1;

FIG. 4 is a side view in partial section of the first embodiment of the safety syringe with the retractable needle hub in FIG. 1;

FIG. 5 is a side view in partial section of the first embodiment of the safety syringe with the retractable needle hub in FIG. 1 showing that a needle is inserted into a syringe body;

FIG. 6 is a side view in partial section of the first embodiment of the safety syringe with the retractable needle hub in FIG. 1 showing that a needle hub is pulled after breaking a bolt;

FIG. 7 is an exploded side view in partial section of the first embodiment of the safety syringe with retractable needle hub in FIG. 1 showing that a plunger is broken at a notch;

FIG. 8 is an exploded side view in partial section of a second embodiment of a safety syringe with a retractable needle hub in accordance with the present invention showing that a plunger is broken at a notch;

FIG. 9 is an exploded perspective view of a third embodiment of a safety syringe with a retractable needle hub in accordance with the present invention;

FIG. 10 is an operational side view in partial section of the third embodiment of the safety syringe with the retractable needle hub in FIG. 9;

FIG. 11 is an exploded side view in partial section of the third embodiment of the safety syringe with the retractable needle hub in FIG. 9 showing that a plunger is broken at a notch;

FIG. 12 is an enlarged perspective view of a first embodiment of a bolt in accordance with the present invention;

FIG. 13 is an enlarged perspective view of a second embodiment of a bolt in accordance with the present invention; and

FIG. 14 is an enlarged perspective view of a third embodiment of a bolt in accordance with the present invention.

### DETAILED DESCRIPTION OF THE PREFERRED EMBODIMENTS

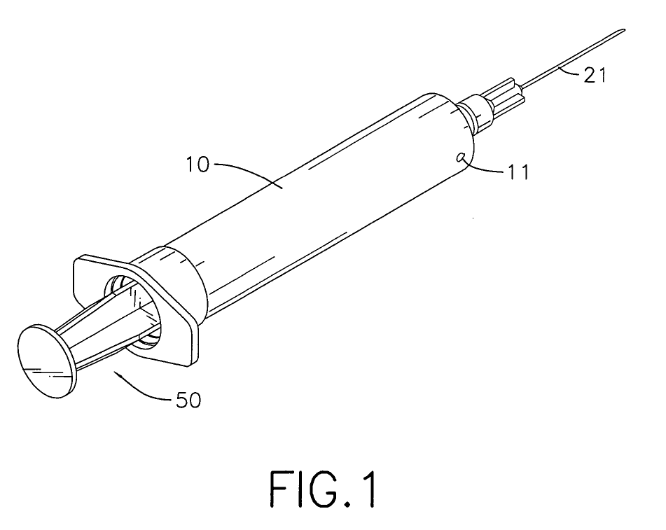

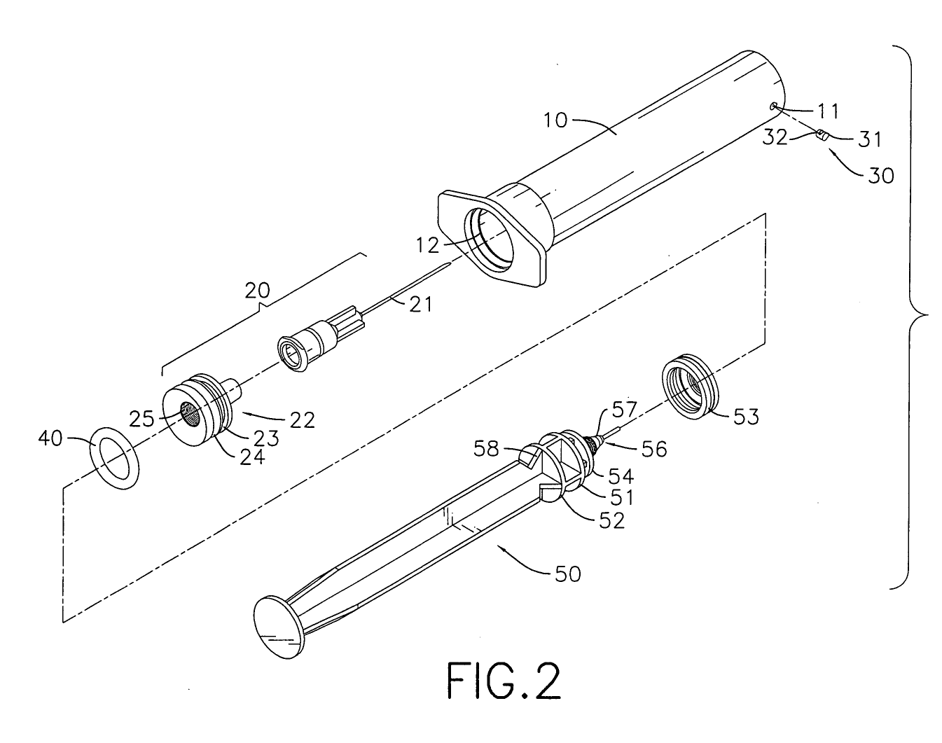

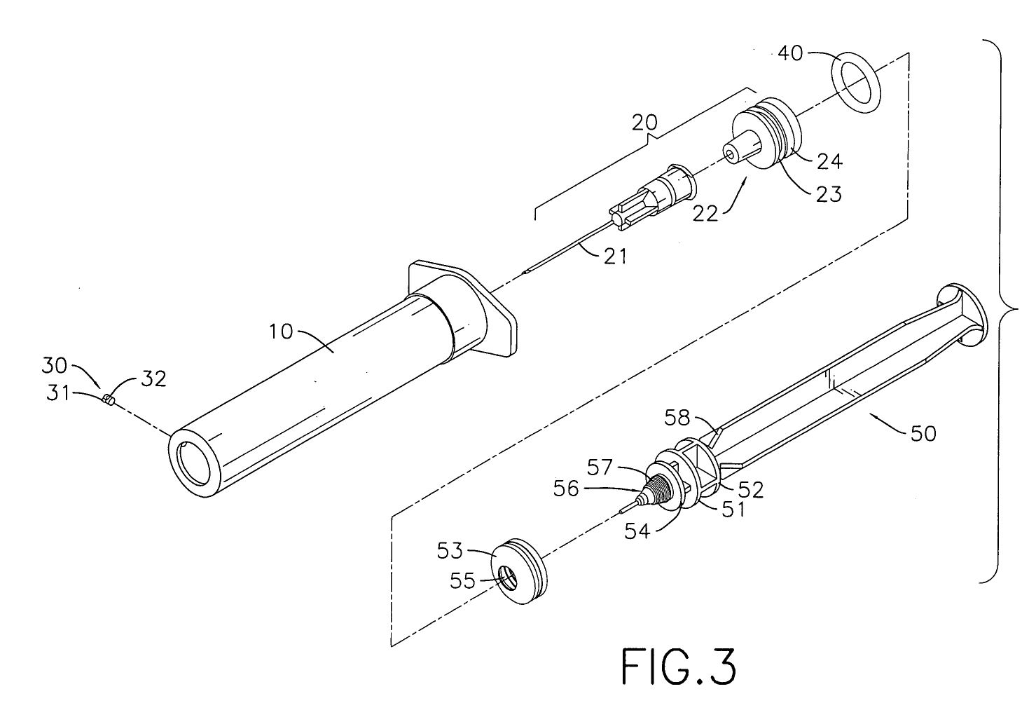

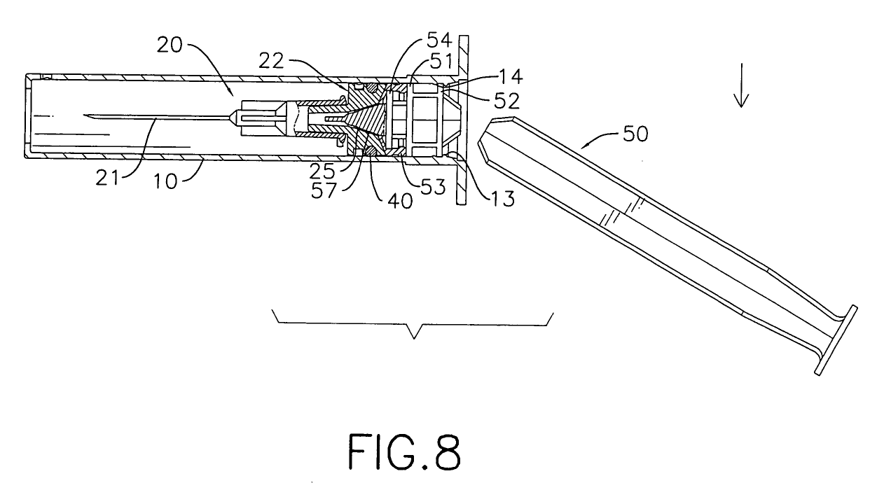

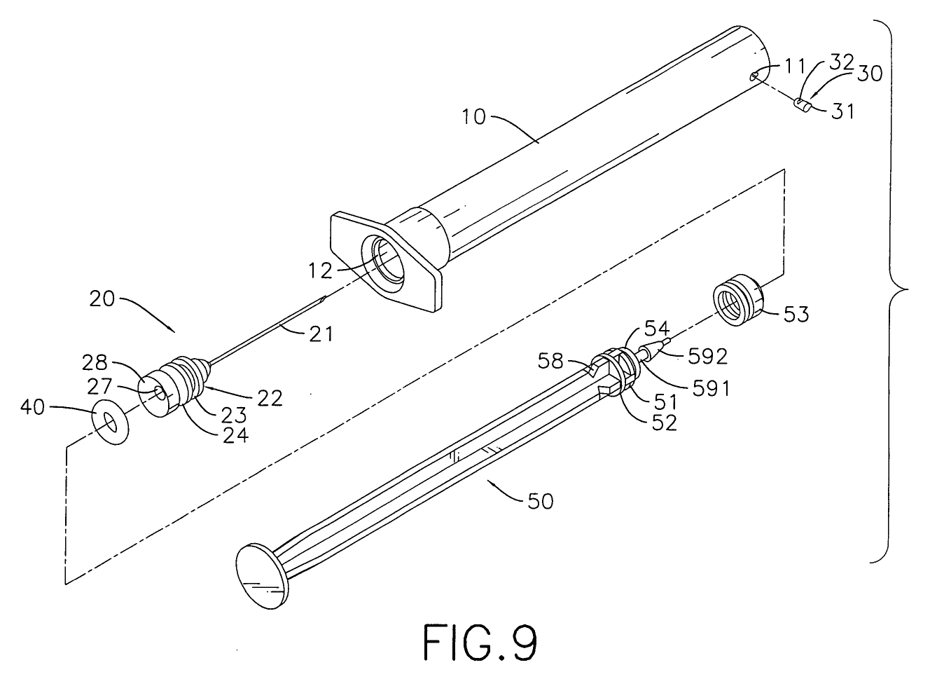

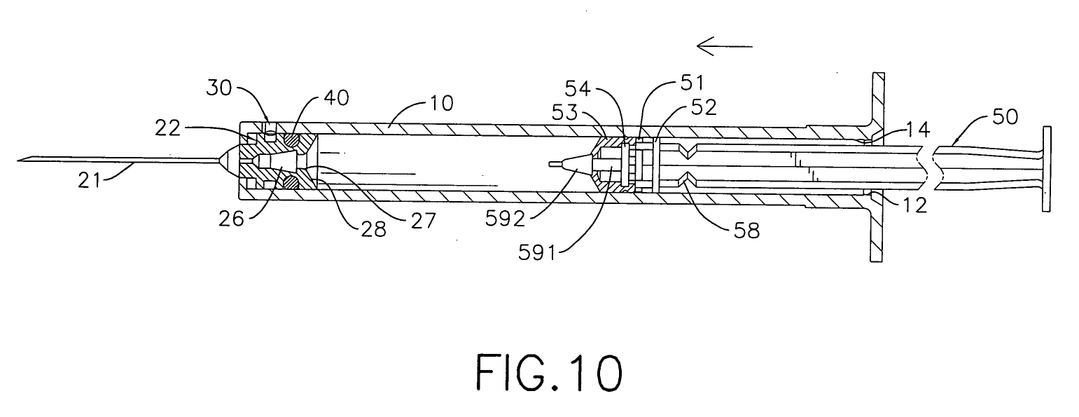

With reference to FIGS. 1 to 3 and 8 to 10, a safety syringe with a retractable needle hub in accordance with the present invention comprises a syringe body (10), a needle hub (20), a bolt (30), an optional ring (40) and a plunger (50).

The syringe body (10) is hollow and comprises a front end, a rear end, an outer surface, a through hole (11), an optional crevice, an inner surface, an inner diameter, a flexible protrusion (14) and a limit (12,13). The through hole (11) is defined through the outer surface of the syringe body (10) near the front end and comprises a circumference. The crevice is formed on the circumference of the through hole (11). The flexible protrusion (14) protrudes on the inner surface of the syringe body (10) near the rear end. The limit (12,13) protrudes on the inner surface of the syringe body (10) between the flexible protrusion (14) and the rear end. The limit (12,13) may be annular or may be multiple protruding dots.

The needle hub (20) is mounted in the syringe body (10) and comprises a base (22) and a needle (21). The base (22) is hollow and comprises a front end, an outer surface, a front recess (23), a rear recess (24), a rear end and a first connector (25,26). The front recess (23) is defined around the outer surface of the base (22) and may be annular. The rear recess (24) is defined around the outer surface of the base (22). The first connector (25,26) is formed in the rear end of the base (22). The first connector (25,26) may be a hole with multiple annular ribs or a hole with a neck (27) and a shoulder (28). The needle (21) is mounted around the front end of the base (22).

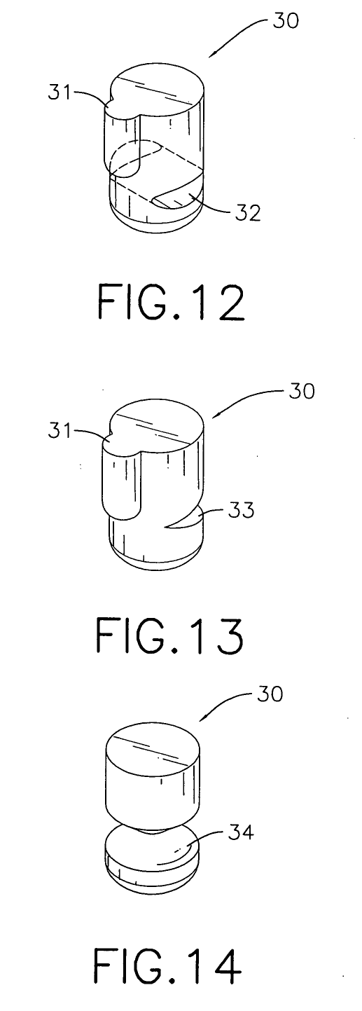

With further reference to FIGS. 12 to 14, the bolt (30) may be cylindrical, is mounted in the through hole (11), extends into the front recess (23) and comprises an outer surface, a cutout (32,33,34) and an optional rib (31). The cutout (32,33,34) is defined in the outer surface of the bolt (30). Preferably, the cutout (32) extends radially through the bolt (30). The cutout (33) may be formed incompletely around the bolt (30) or be annular completely around the bolt (30). The rib (31) is protruded on the outer surface and corresponding with the crevice.

The ring (40) is mounted in the rear recess (24) of the base (22) to prevent leaking.

The plunger (50) is movably mounted in the syringe body (10) and comprises a front end, a front plate (51), an optional rear plate (52), a base (54), a plug (53) and an optional notch (58). The front plate (51) is formed on the front end and comprises a diameter and a front. The diameter of the front plate (51) is corresponding with the inner diameter of the syringe body (10). The rear plate (52) is formed on the plunger (50) separate from the front plate (51) and comprises a diameter. The diameter of the rear plate (52) is corresponding with the inner diameter of the syringe body (10) so the rear plate (52) can pass through the flexible protrusion (14) and hold between the flexible protrusion (14) and the limit (12,13). The base (54) is formed on the front of the front plate (51) and comprises a second connector and a front. The second connector is formed on the front of the base (54) and is selectively fastened with the first connector (25,26). The second connector may be a protrusion (56) with multiple ribs (57) to engage the hole (25) with the ribs. In an alternative embodiment, the second connector may be a rod (591) with a taper head (592) to selectively engage with the hole (26). The plug (53) is mounted around the base (54) and comprises an opening (55) and a center. The opening (55) is defined through the center, and the second connector extends through the opening (55). The notch (58) is defined in the plunger (50) near the rear plate (52).

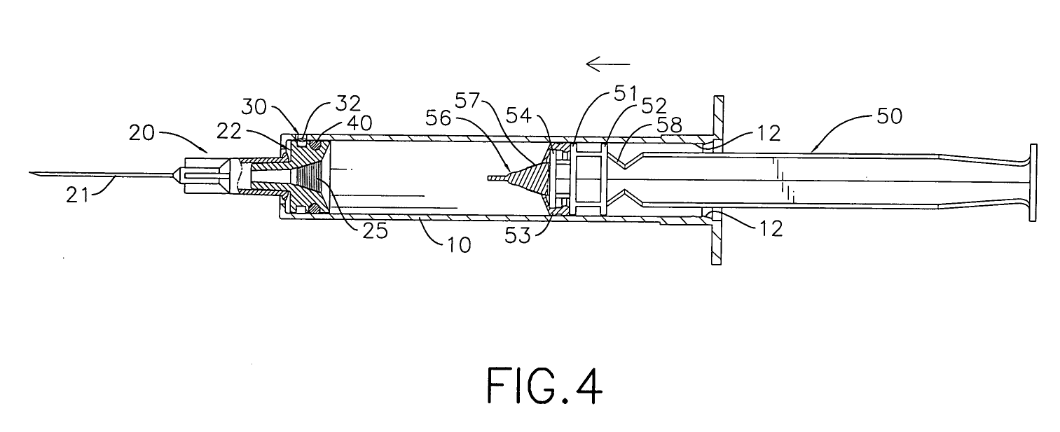

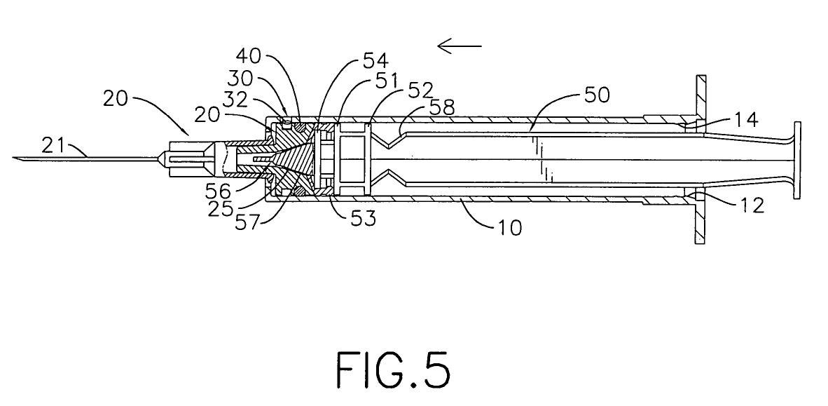

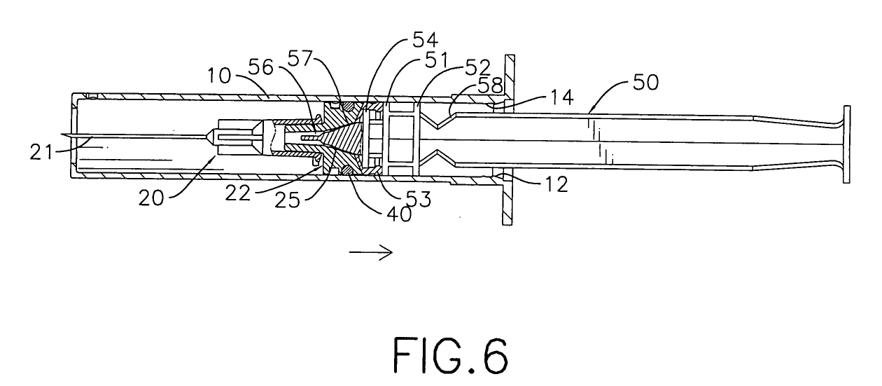

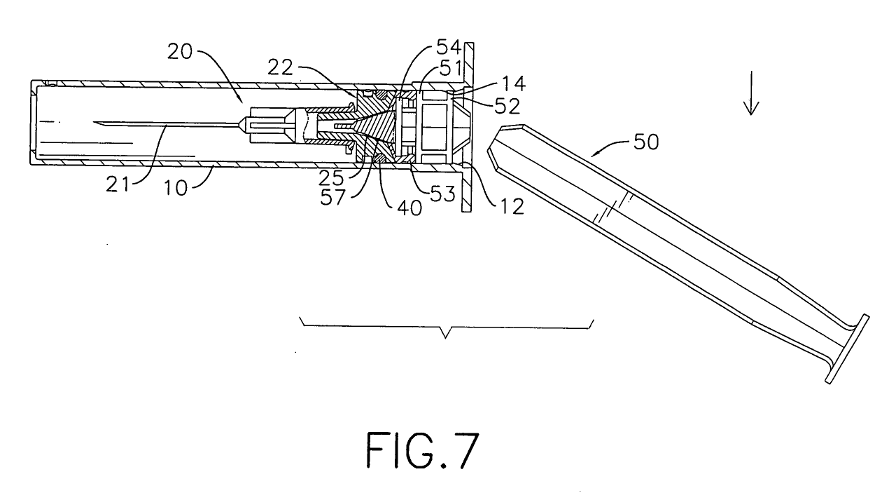

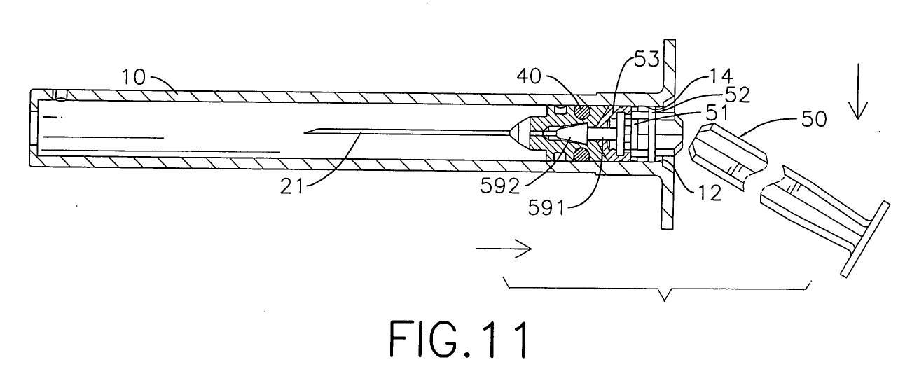

With reference to FIGS. 4 to 7 and 11, the plunger (50) can be pushed into the syringe body (10) to inject solution from the needle (21) or be pulled from the front end to the rear end of the syringe body (10) to suck solution into the syringe body (10). The plunger (50) is pushed into the syringe body (10) to make the second connector engage with the first connector and connect the plunger (50) with the needle hub (20). Then the needle hub (20) can be pushed forward so the movement of the needle hub (20) will break the bolt (30) at the cutout (32, 33, 34). After pulling the plunger (50) backward, the needle hub (20) will move with the plunger (50) and retract into the syringe body (10). When the rear plate (52) passes through the flexible protrusion (14) and abuts the limit (12, 13), the plunger (50) can be broken at the notch (58) so the needle hub (20) with the needle (21) is held inside but cannot be removed from the syringe body (10). Therefore, the used needle will not prick any person.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A safety syringe with a retractable needle hub comprising
a syringe body being hollow and comprising
a front end;
an outer surface;
a through hole defined through the outer surface of the syringe body near the front end and comprising a circumference;
a rear end;
an inner surface;
an inner diameter;
a flexible protrusion protruding on the inner surface of the syringe body near the rear end; and
a limit protruding on the inner surface of the syringe body between the flexible protrusion and the rear end;
a needle hub detachably mounted in the syringe body and comprising
a base being hollow and comprising
a front end;
an outer surface;
a front recess defined around the outer surface of the base;
a rear recess defined around the outer surface of the base;
a rear end; and
a first connector formed in the rear end of the base; and
a needle mounted around the front end of the base;
a bolt mounted in the through hole, extending into the front recess and comprising
an outer surface; and
a cutout defined in the outer surface of the bolt; and
a plunger movably mounted in the syringe body and comprising
a front end;
a front plate formed on the front end and comprising
a diameter corresponding with the inner diameter of the syringe body; and
a front;
a base formed on the front of the front plate and comprising a front; and
a second connector formed on the front of the base and selectively engaging with the first connector; and
a plug mounted around the base of the plunger and comprising a center; and
an opening defined through the center and through which the second connector extends.

2. The safety syringe as claimed in claim 1, wherein the limit is annular.

3. The safety syringe as claimed in claim 1, wherein the limit is multiple protruding dots.

4. The safety syringe as claimed in claim 1, wherein
the first connector is a hole with multiple ribs; and
the second connector is a protrusion with multiple ribs to selectively engage the hole with the ribs.

5. The safety syringe as claimed in claim 1, wherein
the first connector is a hole with a neck and a shoulder; and
the second connector is a rod with a taper head to selectively engage with the hole.

6. The safety syringe as claimed in claim 1, wherein the bolt is cylindrical.

7. The safety syringe as claimed in claim 6, wherein the cutout extends radially through the bolt.

8. The safety syringe as claimed in claim 6, wherein the cutout is formed incompletely around the bolt.

9. The safety syringe as claimed in claim 6, wherein the cutout is annular.

10. The safety syringe as claimed in claim 1, wherein
the syringe body comprises a crevice formed on the circumference of the through hole; and
the bolt comprises a rib protruded on the outer surface and corresponding with the crevice.

11. The safety syringe as claimed in claim 1, wherein the plunger comprises a notch defined in the plunger near the front plate.

12. The safety syringe as claimed in claim 1, wherein the front recess is annular.

13. The safety syringe as claimed in claim 1, wherein the safety syringe with a retractable needle hub comprises a ring mounted in the rear recess of the base to prevent leaking.
